(19) United States
(12) Patent Application Publication
(10) Pub. No.: US 2004/0198809 A1
(43) Pub. Date: Oct. 7, 2004

(54) Use of enantiomeric pure escitalopram

(76) Inventors: Connie Sanchez, Glostrup (DK); Arne Mork, Malov (DK)

Correspondence Address: DARBY & DARBY P.C., P. O. BOX 5257, NEW YORK, NY, 10150-5257, US

(21) Appl. No.: 10/468,685

(22) PCT Filed: May 1, 2002
(86) PCT No.: PCT/DK02/00281

(30) Foreign Application Priority Data
May 1, 2001 (DK) PA 2001-00684

(51) Int. Cl. A61K 31/343

(52) U.S. Cl. 514/469

(57) ABSTRACT

The present invention relates to the use of enantiomeric pure escitalopram and/or of low dose medicaments thereof for the improved treatment of depression, in particular major depression disorder, neurotic disorders, acute stress disorder, eating disorders such as bulimia, anorexia and obesity, phobias, dysthymia, pre-menstrual syndrome, cognitive disorders, impulse control disorders, attention deficit hyperactivity disorder or drug abuse. The medicaments may also be used in the treatment of major depression disorder in “treatment resitant” patients.

The present invention relates to the use of enantiomeric pure escitalopram (INN-name) which is the S-enantiomer of the well-known antidepresssant drug citalopram, i.e. (S)-1-[3-(dimethylamino)propyl]-1-(4-fluorophenyl)-1,3-dihydro-5-isobenzofurancarbonitrile, or a pharmaceutically acceptable salt thereof for the preparation of medicaments, in particular medicaments for the treatment of major depression disorder.

### BACKGROUND OF THE INVENTION

Selective serotonin reuptake inhibitors (hereinafter called SSRIs) such as citalopram have become first-choice therapeutics in the treatment of depression, certain forms of anxiety and social phobias, because they are effective, well-tolerated and have a favourable safety profile compared to the classic tricyclic antidepressants.

However, clinical studies on depression and anxiety disorders indicate that non-response or resistance to SSRIs, i.e. where at least a 40-60% reduction in symptoms has not been achieved during the first 6 weeks of treatment, is substantial, namely up to 30%.

Moreover, there is the delay in therapeutic effect of SSRIs. Sometimes symptoms even worsen during the first weeks of treatment. Even in responders to SSRIs, several weeks of treatment are necessary to achieve a relief in symptoms.

In addition, sexual dysfunction is a side-effect common to all SSRIs.

Without addressing these problems, real progress in the pharmacotherapy of depression and anxiety disorders is not likely to happen.

Escitalopram is the S-enantiomer of the well-known antidepressant drug citalopram and has the following structure:

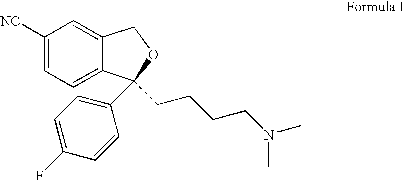

Escitalopram and a method for its preparation are disclosed in U.S. Pat. No. 4,943,590. The stereo selectivity of citalopram, i.e. the 5-HT-reuptake inhibition in the S-enantiomer, and accordingly, its potential antidepressant effect of said enantiomer is also disclosed. It appears that substantially all the 5-HT-reuptake inhibiting effect and accordingly the antidepressant effect is in the S-enantiomer. In view of the stereo-selectivity, escitalopram is expected to be two times as potent as the racemate in the treatment depression.

WO 103694 A1 relates to the use of escitalopram in the treatment of neurotic disorders, including anxiety states and panic attacks.

It has now, surprisingly, been found that the presence of R-citalopram has a negative impact on the effect of escitalopram and escitalopram has been found in pharmacological and clinical studies to be substantially more than two times as potent as the racemate. Furthermore, escitalopram has been found to show a faster onset of action in animal models and clinical studies than the racemate and other SSRIs and to give a more full response in various animal models. Finally, clinical studies have indicated that escitalopram may be an effective medicament in the treatment of depression in patients that do not respond to conventional SSRIs.

The mechanism behind the surprising negative impact of the R-enantiomer on the effect of the S-enantiomer is not known. One possible explanation could be that the R-enantiomer may have a negative influence on the transport of the S-enantiomer over the blood brain barrier. Alternatively, R-citalopram may convey local feed-back inhibition of 5-HT release or the R-enantiomer may modulate the effect of the S-enantiomer.

### DESCRIPTION OF THE INVENTION

Accordingly, the present invention thus relates to the use of escitalopram in low doses and/or comprising less than 3% w/w of R-citalopram for the preparation of a pharmaceutical composition.

In a further aspect, the invention relates to a pharmaceutical composition characterised in that it comprises escitalopram with less than 3% w/w of R-citalopram as an active ingredient.

In yet another aspect, the invention relates to the use of escitalopram for the treatment of major depression disorder characterised in that it is used in a daily dose of less than 10 mg of escitalopram.

As mentioned above, the present invention is based on the finding that R-citalopram has a negative impact on the effect on escitalopram. This may be shown in functional in-vivo pharmacological models and studies of 5-HT-reuptake effect and or in behaviour models, for example depression models.

Escitalopram has also been found to give a significant improvement compared to the double amount of citalopram-racemate and/or to give a more full response. So, it has been found in fixed dose studies that escitalopram in a dose of 10 mg has at least same effect as citalopram in a dose of 40 mg as determined by the MADRS rating scale and Clinical Global Impression (severity as well as improvement).

Escitalopram has also been found in animal models to give a faster response than citalopram-racemate. This has i.a. been found in the Chronic Mild Stress model (Willner P., Psychopharmachology 1997, 134, 319-329). This effect has been confirmed in an 8-week, double-blind, randomised, placebo-controlled, flexible-dose study that compared escitalopram and citalopram to placebo in primary care patients with major depression disorder. The patients received 10 mg escitalopram (155 patients), 20 mg citalopram (160 patients) and placebo (154 patients). Escitalopram showed effects after one week whereas citalopram did not show significant effect.

All these effects are very surprising in view of the prior art suggesting that the R-enantiomer does not influence the effect of the S-enantiomer and, accordingly that escitalopram should only be twice as potent as the racemate.

As a further advantage, the fact that escitalopram is effective in lower doses suggests that effective treatment with less side effects may be obtained, in particular, a reduced amount of serotonin reuptake inhibitor may reduce the risk of SSRI-induced sexual dysfunction and sleep disturbances.

### DETAILED DESCRIPTION OF THE INVENTION

The escitalopram is preferably used as an oxalate salt, preferably a crystalline oxalate salt.

Furthermore, in the escitalopram used, R-citalopram is preferably not present in an amount exceeding 2% w/w, most preferably 1% w/w. The percentage of R-citalopram is throughout the description given as w/w % compared to the amount of escitalopram present.

The pharmaceutical composition of the invention is preferably for the treatment of depression, in particular major depression disorder, neurotic disorders, acute stress disorder, eating disorders such as bulimia, anorexia and obesity, phobias, dysthymia, premenstrual syndrome, cognitive disorders, impulse control disorders, attention deficit hyperactivity disorder or drug abuse.

Throughout this specification and claims the term “neurotic disorders” is used to designate a group of mental disorders, including anxiety states, in particular generalised anxiety disorder and social anxiety disorder, post traumatic stress disorder, obsessive compulsive disorder and panic attacks.

The terms “generalised anxiety disorder”, “social anxiety disorder”, “post traumatic stress disorder” and “obsessive compulsive disorder” are as defined in DSM IV.

The phrase “panic attacks” contemplates treatment of any disease, which is associated with panic attacks including panic disorder, specific phobias, social phobia and agoraphobia in which panic attacks occur. These disorders are further defined in the DSM IV.

The phrase “treatment of panic disorder” means a reduction in the number or prevention of attacks and/or relief of the severity of the attacks. Similarly, the treatment of generalised anxiety disorder, social anxiety disorder, post traumatic stress disorder and obsessive compulsive disorder include the treatment or prevention of these diseases, or the relief of the symptoms thereof.

Based on the pharmacological and clinical studies, preferred indications are major depression disorder and obsessive compulsive disorder.

Other preferred uses are treatment of neurotic disorders.

In particular, the composition may be useful for treatment of patients who have failed to respond to initial treatment with a conventional SSRI, in particular patients with major depression disorder who have failed to respond to initial treatment with a conventional SSRI. Such treatment resistant patients may in particular be defined a patients who do not achieve an alleviation in symptoms of 40-60% by treatment with citalopram or other marketed SSRIs. Further definitions are given in Kornstein S C and Schneider R K, Clinical features of treatment-resistant depression J Clin Psychiatr 2001, 62, Suppl 16, 18-25; Sackeim H A, The definition and meaning of treatment-resistant depression, J. Clin Psychiatr 2001, 62 Suppl 16, 10-17; and Nierenber A A and DeCecco L M, Definitions of antidepressant treatment response, remission, non-response, partial response, and other relevant outcomes: A focus on treatment-resistant depression J Clin Psychiatr 2001, 62 Suppl 16, 5-9.

The pharmaceutical composition according to the invention may comprise escitalopram in a unit dose preparation containing 2.5 to 20 mg escitalopram.

In view of the potent effect of the escitalopram used according to the invention, it may be effective in low doses, i.e. daily doses lower than 10 mg escitalopram, for example 7.5 mg or lower, such as 7.5 or 5 mg pr day.

The pharmaceutical composition according to the invention is preferably an oral formulation, preferably a tablet.

Thus, tablets may be prepared by mixing the active ingredient with ordinary adjuvants and/or diluents and subsequently compressing the mixture in a conventional tabletting machine. Examples of adjuvants or diluents comprise: corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

Solutions for injections may be prepared by dissolving the active ingredient and possible additives in a part of the solvent for injection, preferably sterile water, adjusting the solution to desired volume, sterilisation of the solution and filling in suitable ampules or vials. Any suitable additive conventionally used in the art may be added, such as tonicity agents, preservatives, antioxidants, etc.

Clinical Study

A total of 471 patients were randomised into the study. The all-patient-treated set comprised 469 patients and the fill-analysis set comprised 468 patients. In the fill-analysis set there were 155 patients in the escitalopram group, 159 patients in the citalopram group, and 154 patients in the placebo group.

There was an approximately 3 to 1 ratio of women to men in each treatment group, and almost all patients were Caucasian. The mean age was 43 years (SD 11). At baseline, the mean MADRS total score was approximately 29 for the treatment group, which signifies moderate to severely ill patients.

The efficacy analysis of the adjusted mean change in MADRS total score showed a significantly superior therapeutic effect for escitalopram versus placebo from Week 1 (p=0.023) to Week 4 (p=0.002) ) (observed cases). At Week 4, the adjusted mean change in

MADRS total score (last observation carried forward) for escitalopram versus placebo was 2.7 points >(p=0.002) compared to a statistically insignificant change of 1.5 points for citalopram versus placebo.

Escitalopram was significantly superior to placebo both on the CGI improvement and severity subscale from Week 1 (p<0.05)(observed cases) onwards, while citalopram was not statistically different from placebo during the 4-week period. At Week 4 (last observation carried forward), escitalopram was statistically significantly superior to placebo while there was no statistically significant difference between citalopram versus placebo.

1-19. (canceled)

20: A unit dose preparation comprising 7.5 mg or less of escitalopram or a pharmaceutically acceptable salt thereof.

21: The unit dose preparation of claim 20, wherein the unit dose preparation comprises 7.5 mg of escitalopram or a pharmaceutically acceptable salt thereof.

22: The unit dose preparation of claim 20, wherein the unit dose preparation comprises 5 mg of escitalopram or a pharmaceutically acceptable salt thereof.

23: The unit dose preparation of claim 20, wherein the unit dose preparation comprises 2.5 mg of escitalopram or a pharmaceutically acceptable salt thereof.

24: The unit dose preparation of claim 20, wherein the unit dose preparation is a tablet.

25: The unit dose preparation of claim 21, wherein the unit dose preparation is a tablet.

26: The unit dose preparation of claim 22, wherein the unit dose preparation is a tablet.

27: The unit dose preparation of claim 23, wherein the unit dose preparation is a tablet.

28: The unit dose preparation of claim 20, wherein the pharmaceutically acceptable salt is an oxalate salt.

29: The unit dose preparation of claim 21, wherein the pharmaceutically acceptable salt is an oxalate salt.

30: The unit dose preparation of claim 22, wherein the pharmaceutically acceptable salt is an oxalate salt.

31: The unit dose preparation of claim 23, wherein the pharmaceutically acceptable salt is an oxalate salt.

32: The unit dose preparation of claim 28, wherein the pharmaceutically acceptable salt is a crystalline oxalate salt.

33: The unit dose preparation of claim 29, wherein the pharmaceutically acceptable salt is a crystalline oxalate salt.

34: The unit dose preparation of claim 30, wherein the pharmaceutically acceptable salt is a crystalline oxalate salt.

35: The unit dose preparation of claim 31, wherein the pharmaceutically acceptable salt is a crystalline oxalate salt.

36: A method of treating major depression disorder in a patient in need thereof, the method comprising administering the unit dose preparation of claim 20 to the patient.

37: A method of treating major depression disorder in a patient in need thereof, the method comprising administering the unit dose preparation of claim 21 to the patient.

38: A method of treating major depression disorder in a patient in need thereof, the method comprising administering the unit dose preparation of claim 22 to the patient.

39: A method of treating major depression disorder in a patient in need thereof, the method comprising administering the unit dose preparation of claim 23 to the patient.

40: A method of treating major depression disorder in a patient in need thereof, the method comprising administering a pharmaceutically effective amount of escitalopram or a pharmaceutically acceptable salt thereof to the patient, wherein the pharmaceutically effective amount is a daily dose of 10 mg or less of escitalopram or a pharmaceutically acceptable salt thereof.

41: The method of claim 40, wherein the daily dose is 7.5 mg or less of escitalopram or a pharmaceutically acceptable salt thereof.

42: The method of claim 41, wherein the daily dose is 7.5 mg of escitalopram or a pharmaceutically acceptable salt thereof.

43: The method of claim 41, wherein the daily dose is 5 mg of escitalopram or a pharmaceutically acceptable salt thereof.

44: The method of claim 41, wherein the daily dose is 2.5 mg of escitalopram or a pharmaceutically acceptable salt thereof.

45: A method of treating major depression disorder in a patient in need thereof, the method comprising administering a daily pharmaceutically effective amount of escitalopram or a pharmaceutically acceptable salt thereof to the patient, wherein the daily dose is 10 mg or less of escitalopram or a pharmaceutically acceptable salt thereof and the daily dose is administered to obtain an effect in the patient after one week.

46: The method of claim 45, wherein the daily dose is 10 mg of escitalopram or a pharmaceutically acceptable salt thereof.
